SEQUENCE LISTING (6 sequences)

SEQ ID NO: 1 - DNA, 19 bp, Artificial
  Bridge linker forward
  misc_feature (10)..(10): Biotin-labeled
cgcgatatcttatctgact

SEQ ID NO: 2 - DNA, 19 bp, Artificial
  Bridge linker reverse
gtcagataagatatcgcgt

SEQ ID NO: 3 - DNA, 33 bp, Artificial
  Adaptor forward
gatcggaagagcacacgtctgaactccagtcac

SEQ ID NO: 4 - DNA, 34 bp, Artificial
  Adaptor reverse
tacactctttccctacacgacgctcttccgatct

SEQ ID NO: 5 - DNA, 45 bp, Artificial
  Common primer
aatgatacggcgaccaccgagatctacactctttccctacacgac

SEQ ID NO: 6 - DNA, 51 bp, Artificial
  Index primer
caagcagaagacggcatacgagatcgtgatgtgactggagttcagacgtgt

(19) United States
(12) Patent Application Publication
(10) Pub. No.: US 2021/0010062 A1
(43) Pub. Date: Jan. 14, 2021

(54) METHOD FOR ANALYZING AN INTERACTION EFFECT OF NUCLEIC ACID SEGMENTS IN NUCLEIC ACID COMPLEX

(71) Applicant: TSINGHUA UNIVERSITY, Beijing (CN)

(72) Inventors: Yang CHEN, Beijing (CN); Zhengyu LIANG, Beijing (CN); Yanjian LI, Beijing (CN); Guipeng LI, Beijing (CN); Qiwei ZHANG, Beijing (CN)

(73) Assignee: TSINGHUA UNIVERSITY, Beijing (CN)

(21) Appl. No.: 16/944,185

(22) Filed: Jul. 31, 2020

Related U.S. Application Data
(63) Continuation of application No. PCT/CN2018/112331, filed on Oct. 29, 2018.

(30) Foreign Application Priority Data
Oct. 27, 2017 (CN) 201711024711.2

(51) Int. Cl. C12Q 1/6806; C12Q 1/6869; C12N 15/10

(52) U.S. Cl. CPC C12Q 1/6806; C12N 15/1093; C12Q 1/6869

(57) ABSTRACT

Provided is a method of analyzing interactions between nucleic acid segments in a nucleic acid complex. Specifically, restriction enzymes that recognize four-base site are used for digestion, followed by a two-step ligation method. The overall process is simple and easy to control, realizing the efficient and sensitive detection of nucleic acid interaction segments.

### CROSS REFERENCE TO RELATED APPLICATIONS

This application claims the priority of Chinese Patent Application No. 201711024711.2, filed on Oct. 27, 2017, and the disclosures of which are hereby incorporated by reference.

### FIELD

The present disclosure belongs to the field of nucleic acid interaction analysis, and relates to a method of analyzing interactions between nucleic acid segments in three dimensions in a nucleic acid complex.

### BACKGROUND

After years of research, people's understanding of the three-dimensional structure of chromatin has gradually deepened, including the gradual folding of DNA to form chromatin fibers, topologically associating domains (TADs), and active/inactive compartments (AB compartment). The establishment of large-scale chromatin structures such as topological domains in the early embryonic development of mammals and the dynamic changes in the cell cycle have been studied. More and more evidences show that, in the delicate chromatin structure, structure proteins and transcription factors play important roles in maintaining chromatin interaction and regulating chromatin conformational changes. In order to directly capture and explore such delicate chromatin interactions, people have developed high-throughput chromosome conformation capture (Hi-C) and a variety of Hi-C deformation techniques, mainly divided into two major classes. One type is based on the Chromatin Immunoprecipitation (ChIP) technique, of which the principle is to use antibodies to capture chromatin interactions mediated by specific proteins, such as ChIA-PET (Chromatin Interaction Analysis by Paired-End Tag Sequencing) and HiChIP. However, this type of method requires the use of up to one million cells and specific antibodies for enrichment, making it difficult to apply to a system with small number of cells and transcription factors. Another method is based on probes capturing and enriching specific DNA sequences to obtain chromatin structures that interact with the sequences, such as Capture Hi-C. However, this type of method requires designing probes for known DNA sites, which greatly reduces the discrimination of similar sequences. Due to the inherent defects of the above techniques, there is an urgent need for a simpler and more efficient method for the study of nucleic acid interactions in nucleic acid complexes with complicated structures.

### SUMMARY

The object of the present disclosure is to provide a more efficient and sensitive method for detecting nucleic acid complex interactions, particularly chromatin interactions, and nucleic acid segment interactions in chromatin. The applicant has unexpectedly found that when the restriction enzyme HaeIII is used to replace the traditional MboI enzyme for chromatin fragmentation, although HaeIII, which recognizes the four-base sequence GGCC, cleaves the human genome and the average fragment length is 342 bp, which is close to the average fragment length of 401 bp produced by the MboI enzyme used in traditional Hi-C, but the distances between the cleavage site of HaeIII and the binding proteins (such as RNAPII, CTCF, or DNase) are significantly shorter than that of MboI, which greatly facilitates the separation and identification of the DNA sequences bound by the binding protein, and the efficiency far exceeds the traditional Hi-C method. Not only that, the applicant also creatively introduced bridge linkers for the ligation of the adjacent DNA fragments after digestion, which greatly increased the ligation probability of DNA fragments inside the “protein-DNA” complex and significantly increased the amount of protein-mediated chromatin, to the greatest extent, excludes the false positive results from the ligation between DNAs without binding.

In the first aspect, the present disclosure provides a method of analyzing interactions between two or more nucleic acid segments in a nucleic acid complex, comprising

1) providing a sample comprising the nucleic acid complex;

2) exposing the nucleic acid complex obtained in step 1) to a restriction enzyme of which the recognition site is located in or near at least one of the nucleic acid segments, and performing digestion;

3) subjecting the resultant of the digestion from step 2) to ligation; and

4) identifying the sequences of the two or more nucleic acid segments which are ligated in step 3).

In one embodiment, step 1) includes performing a cross-linking treatment on the sample, and the cross-linking treatment is preferably performed using a cross-linking agent.

Specifically, the cross-linking agent is preferably glutaraldehyde, formaldehyde, epichlorohydrin and toluene diisocyanate, more preferably formaldehyde.

Optionally, the crosslinking is in situ cross-linking.

In another embodiment, the two or more nucleic acid segments are genetic regulatory sequences, preferably, the genetic regulatory sequences are promoter, silencer and enhancer.

In another embodiment, the two or more nucleic aide segments are bound to one or more binding proteins, which are preferably selected from transcription factor, enhancer binding protein, RNA polymerase and CTCF.

In another embodiment, the restriction enzyme is preferably a restriction enzyme with a recognition site of four-base sequence, more preferably a restriction enzyme with a recognition site of GGCC and/or CCTC, and most preferably HaeIII or MnlI.

In one embodiment, the ligation in step 3) is performed by using bridge linker to link the nucleic aide segments (for example, segments that are close), and the bridge linker refers to an adaptor sequence that links the terminals of different nucleic aide fragments.

In one embodiment, the bridge linker is a double-stranded nucleic acid.

The length of the bridge linker is preferably 10-60 bp, 15-55 bp, 20-50 bp, 25-45 bp or 30-40 bp, such as 15 bp, 16 bp, 17 bp, 18 bp, 19 bp, 20 bp, 21 bp, 22 bp, 23 bp, 24 bp, 25bp, 26 bp, 27 bp, 28 bp, 29 bp, 30 bp, 31 bp, 32 bp, 33 bp, 34 bp or 35 bp, more preferably 20 bp.

In one embodiment, the bridge linker may be labeled with one or more markers, preferably, the marker includes biotin, fluorescein and antibody, more preferably biotin.

In one embodiment, the marker is labeled at the 5′ terminal, 3′ terminal or middle region of the bridge linker.

In one embodiment, the marker may be labeled in any one strand or both strands of the double-stranded nucleic acid.

In one embodiment, the identification of ligated sequences in step 4) is performed by sequencing, preferably, the sequencing is Sanger sequencing, second generation sequencing, single molecule sequencing and single cell sequencing, more preferably second generation sequencing

In one embodiment, upon the identification of ligated sequences in step 4), the method further comprises steps of de-crosslinking, nucleic acid purification, fragmentation (e.g. by sonication), enrichment, library construction and/or PCR amplification.

In another aspect, the present disclosure provides a method of analyzing interactions between one or more genetic regulatory sequences of interest and other nucleic aide segments, comprising the steps of any one method of the first aspect.

In another aspect, the present disclosure provides a method of identifying nucleic aide sequence interacting with one or more genetic regulatory sequences of interest, comprising the steps of any one method of the first aspect.

In another aspect, the present disclosure provides a method of determining the expression state of a target gene, comprising the steps of any one method of the first aspect, and analyzing the state, type and density of interactions between regulatory sequences of the target gene and other nucleic aide segments.

In another aspect, the present disclosure provides a method of changing the expression state of a target gene, comprising the steps of any one method of the first aspect, and changing the state, type and density of interactions between regulatory sequence segments of the target gene and other nucleic aide segments.

In another aspect, the present disclosure provides a method of identifying an agent capable of regulating the expression of a target gene, comprising contacting a sample with one or more agents, analyzing interactions related to the expression regulation of the target gene between two or more nucleic aide segments using the steps of any one method of the first aspect, and identifying the agent capable of changing the interaction when comparing to a control sample without the agent.

In another aspect, the present disclosure provides a method of analyzing higher-order structure of genetic material, comprising the steps of any one method of the first aspect.

In another aspect, the present disclosure provides a method of identifying structure changes of chromatin, comprising the steps of any one method of the first aspect.

In another aspect, the present disclosure provides a method of identifying a regulatory agent for higher-order structure of genetic material, comprising contacting a sample with one or more regulatory agents, analyzing interactions between two or more nucleic aide segments using the steps of any one method of the first aspect, and identifying the regulatory agent capable of changing the interaction of nucleic aide segments when comparing to a control sample without the regulatory agent.

In another aspect, the present disclosure provides a method of constructing a sequencing library for chromatin interaction analysis, comprising steps 1) to 3) of any one method of the first aspect, followed by step 5) releasing the linked segments, to construct the sequencing library.

In another aspect, the present disclosure provides a method of identifying a nucleic aide-protein complex, comprising the steps of any one method of the first aspect, and identifying the nucleic aide-protein complex according to the results of nucleic aide segment interactions and information of binding between the nucleic aide segments and the proteins.

In another aspect, the present disclosure provides a method of identifying a protein-protein complex, comprising the steps of any one method of the first aspect, and identifying the protein-protein complex according to the results of nucleic aide segment interactions and information of binding between the nucleic aide segments and the proteins.

In another aspect, the present disclosure provides a method of identifying interactions between gene transcription regulatory sequences, comprising the steps of any one method of the first aspect, and analyzing the type, number and/or density of nucleic aide segment interactions in promoter and enhancer regions.

In another aspect, the present disclosure provides a method of determining the stability of chromatin topologically associating domain (TAD) boundary, comprising the steps of any one method of the first aspect, and analyzing the type, number and/or density of interactions between CTCG binding nucleic aide segments.

In another aspect, the present disclosure provides a method of genome mapping, comprising sequencing and the steps of any one method of the first aspect, and using the interaction information of nucleic aide segments to assist the localization and mapping of the sequences.

In another aspect, the present disclosure provides a method of identifying one or more nucleic aide interactions related to a specific disease, comprising the steps of any one method of the first aspect, wherein in step 1), samples from a patient and a healthy person are provided, and the interactions showing different may be used to indicate the specific disease; preferably, the disease is a genetic disease or cancer.

In another aspect, the present disclosure provides a method of diagnosing a disease related to structural changes of chromatin, comprising the steps of any one method of the first aspect, wherein in step 1), samples from a subject is provided, and the diagnosis is based on the results of nucleic aide segment interactions; preferably, the disease is a genetic disease or cancer.

In another aspect, the present disclosure provides a kit used for using in any of one of the methods of the aspects above.

In another aspect, the present disclosure provides a kit, comprising a restriction enzyme capable of recognizing GGCC and/or CCTC sites and/or bridge linkers, wherein

the restriction enzyme is capable of recognizing four bases site, preferably a restriction enzyme capable of recognizing CCTC and/or GGCC sites, more preferably HaeIII or MnlI;

the length of the bridge linker is 10-60 bp, 15-55 bp, 20-50 bp, 25-45 bp or 30-40 bp, such as 15 bp, 16 bp, 17 bp, 18 bp, 19 bp, 20 bp, 21 bp, 22 bp, 23 bp, 24 bp, 25bp, 26 bp, 27 bp, 28 bp, 29 bp, 30 bp, 31 bp, 32 bp, 33 bp, 34 bp or 35 bp, preferably 20 bp;

the bridge linker may be labeled a marker, preferably, the marker preferably includes isotopes, biotin, digoxin (DIG), fluorescein (such as FITC and rhodamine) and/or a probe, more preferably biotin;

the marker is labeled at the 5′ terminal, 3′ terminal or middle region of the bridge linker; and

the kit is a kit for sequencing or library construction.

In another aspect, the present disclosure provides use of the restriction enzyme capable of recognizing GGCC and/or CCTC sites, or the kit for

1) analyzing interactions between one or more nucleic aide segments in a nucleic aide complex;

2) analyzing interactions between one or more genetic regulatory sequences of interest and other nucleic aide segments;

3) identifying nucleic aide sequence interacting with one or more genetic regulatory sequences of interest;

4) determining the expression state of a target gene;

5) changing the expression state of a target gene;

6) changing the interactions between regulatory elements of target gene and other nucleic aide sequence;

7) analyzing higher-order structure of genetic material;

8) identifying structure changes of chromatin;

9) identifying a regulatory agent for higher-order structure of genetic material;

10) constructing a sequencing library for chromatin interaction analysis;

11) identifying a nucleic aide-protein complex;

12) identifying a protein-protein complex;

13) identifying interactions between gene transcription regulatory sequences;

14) determining the stability of chromatin topologically associating domain (TAD) boundary;

15) identifying an agent capable of regulating the expression of a target gene;

16) genomic mapping;

17) identifying one or more nucleic aide interactions indicating a specific disease; and

18) diagnosing a disease related to structural changes of chromatin;

19) preparing a kit for diagnosing a disease related to structural changes of chromatin; and

20) preparing a kit for identifying one or more nucleic aide interactions related to a specific disease.

In another aspect, the present disclosure provides a bridge linker for the method of any one method of the above aspects, wherein

the bridge linker is preferably a double-stranded nucleic acid;

the nucleic acid may be labeled with one or more markers at the 5′ terminal, 3′ terminal or middle region thereof, preferably, the marker is isotopes, biotin, digoxin (DIG), fluorescein (such as FITC and rhodamine) and probe, more preferably biotin;

the length of the nucleic acid is 10-60 bp, 15-55 bp, 20-50 bp, 25-45 bp or 30-40 bp, such as 15 bp, 16 bp, 17 bp, 18 bp, 19 bp, 20 bp, 21 bp, 22 bp, 23 bp, 24 bp, 25bp, 26 bp, 27 bp, 28 bp, 29 bp, 30 bp, 31 bp, 32 bp, 33 bp, 34 bp or 35 bp, preferably 20 bp; and

specifically, the marker is labeled at the 5′ terminal, 3′ terminal or middle region of the nucleic acid, specifically, the marker may be labeled in any one strand or both strands of the double-stranded nucleic acid.

The summary of the present disclosure only exemplifies some specific embodiments, wherein the technical features described in one or more technical solutions can be combined with any one or more technical solutions, and these combined technical solutions are also within the scope of this invention.

In the methods of the present disclosure, by using a specific four-base recognition enzyme, making the recognition site closer to the nucleic acid sequences of interest, for example, nucleotide segments that interact with the CTCF maintaining the chromatin loop or active transcription factor. The biotin-labeled dCTP (Biotin-14-dCTP) used in traditional in situ Hi-C is replaced by a bridge linker, since the biotin labeling in the bridge linker only needs to be modified during the synthesis of the nucleic acid, it can be achieved by ordinary biotechnology companies, greatly reducing the cost. In situ Hi-C, Biotin-14-dCTP needs to be added during the terminal blunting process, and the related reagents are very expensive. Therefore, the methods of the present invention can reduce the cost to one-third of the original. The methods of the present invention have broad applications in study the interactions of nucleic acid segments in nucleic acid complexes, such as chromatin interaction, drug screening, and diagnosis of chromatin-related diseases.

### BRIEF DESCRIPTION OF DRAWINGS

The present disclosure will be clearly explained by the detailed specification and the accompanying drawings. In order to illustrate the present invention, the embodiments in the drawings are preferred embodiments, however, it should be understood that the present invention is not limited to the specific embodiments here.

FIG. 1-A shows the overall flowchart of the BL-Hi-C method.

FIG. 1-B shows the comparison of BL-Hi-C, in situ Hi-C and HiChIP on paired-end tags (PETs) numbers.

FIG. 2-A shows the comparison of BL-Hi-C, in situ Hi-C and HiChIP on CTCF and POL2A peaks.

FIG. 2-B shows the distribution of reads detected by BL-Hi-C in promoters, enhancers and heterochromatin regions, indicating that BL-Hi-C detects more interactions close to active promoters and strong enhancers, and less than 50% of the reads are located in the heterochromatin region.

FIG. 2-C shows the enrichment of BL-Hi-C reads at transcription factor-binding sites.

FIG. 2-D shows the relative ratio of CTCF peaks obtained by BL-Hi-C or in situ Hi-C.

FIG. 2-E shows the enrichment of high, normal, and low grouped CTCF peaks at genome. It can be seen that most of the peaks are in the promoter region, not introns or intergenic regions.

FIG. 3-A shows the percentages of CTCT peaks and RNAP II peaks in PETs obtained by BL-Hi-C or in situ Hi-C.

FIG. 3-B shows the percentage comparison of peaks in PETs obtained by BL-Hi-C or in situ Hi-C.

FIG. 3-C shows the relative ratio of RNAP II peaks obtained by BL-Hi-C or in situ Hi-C.

FIG. 3-D shows the enrichment of high, normal, and low grouped RNAP II peaks at genome. It can be seen that most of the peaks are in the promoter region, not introns or intergenic regions.

FIG. 4 shows the comparison of enzymes and ligation methods. FIG. 4-A shows the comparison results from the digestion with HaeIII, MboI and HindIII, respective; FIG. 4-B shows the comparison results when using one-step ligation and two-step ligation.

FIG. 5-A shows the comparison of statistical analysis of the distance between the restriction sites of HaeIII, MboI and HindIII and different binding proteins.

FIG. 5-B shows the theoretical models of one-step ligation and two-step ligation.

FIG. 5-C shows SNR simulation calculation results of one-step ligation and two-step ligation.

FIG. 6-A shows the chromatin loops determined by combined data sets from BL-Hi-C and in situ Hi-C.

FIG. 6-B shows the percentages of common loops and specific loops that are consistent with the public ChIA-PET loops of CTCF.

FIG. 6-C shows the percentages of common loops and specific loops that are consistent with the public ChIA-PET loops of RNAPII.

FIG. 6-D shows comparison of ChIA-PET loops and Hi-C loops in a typical region, chromosome 12.

FIG. 6-E shows the normalized PET counts of the loops identified by BL-Hi-C and in situ Hi-C.

FIG. 6-F shows the normalized interaction heatmaps of BL-Hi-C (left), in situ Hi-C, and the difference (right) at 10 kb resolution (up) and 1 kb resolution (down) of chromosome 11.

FIG. 6-G shows the chromatin interaction detection results of visual 4C on β-globin region.

FIG. 7 shows the verification of chromatin loops determined by BL-Hi-C using 4C-seq technique.

FIG. 8 shows the average distribution comparison of different 4-base pair recognition sites in human genome and mouse genome.

FIG. 9 shows the comparison of distance between different four-base pair recognition sites and promoters and enhancers in the genome.

FIG. 10 shows the frequency of four-base pair recognition sites within five hundred bases of different transcription factor binding sites in the K562 cell line.

### DETAILED DESCRIPTION

The terms used in this application have the same meaning as the terms in the prior art. In order to clearly indicate the meaning of the terms used, the specific meanings of some terms in this application are given below. When the definition in this application conflicts with the conventional meaning of the term, the definition in this application shall prevail.

The term “nucleic acid complex” refers to a complex with a certain spatial structure formed by at least the participation of nucleic acids, and the spatial structure contains higher-order structures of nucleic acids, such as loops and folded structures. The nucleic acid complex may be composed only of nucleic acids, such as DNA or RNA with a higher-order structure, or may additionally contain other molecules, such as proteins. Therefore, from a broad perspective, the nucleic acid complex in the present invention also includes the concept of nucleic acid-protein complex; specifically, chromatin (“chromatin” in the present invention can also be replaced with “chromosome”) belongs to a kind of nucleic acid complex.

The most abundant protein in chromatin is histone. The structure of chromatin depends on several factors, and the overall structure depends on the stage of the cell cycle. During the interphase, the structure of chromatin is loose, allowing the approach of RNA polymerases and DNA polymerases that transcribe and replicate DNA. The local structure of the chromatin in the interphase depends on the genes on the DNA: genes encoding DNA that are actively transcribed are the most loose, and they are binding with RNA polymerases, called euchromatin; whereas DNA encoding inactive genes is binding with structural proteins and more tightly packed, called heterochromatin. Epigenetic modifications of structural proteins in chromatin also change local chromatin structure, especially chemical modification of histones by methylation and acetylation. When cells are ready to divide, that is, into mitosis or meiosis, chromatin is more tightly packed to promote chromosome segregation in the later stages of division. In the nucleus of eukaryotic cells, different parts of the chromosome have unique chromosomal regions during interphase. Recently, large megabase-sized local chromatin interaction domains have been identified, called “topologically associating domain (TAD)”, which are associated with genomic regions that constrain heterochromatin diffusion. The domains are stable in different cell types and are highly conserved among species. On the one hand, they interact with each other, and on the other hand, they provide a basis for the formation of higher-order structures in the genome. The method of the present invention is suitable for analyzing chromatin structure and its interaction.

The term “nucleotide segment” or “nucleotide fragment” refers to a continuous sequence formed by nucleotides (such as deoxyribonucleotide), which may exist independently or may be located in a longer nucleic acid sequence.

The term “two or more nucleic acid segments” refers to nucleic acid segments/fragments located in different regions of the nucleic acid complex. The analyzed nucleic acid segments may not be the target sequences, or part of the target sequence, or all the nucleic acid sequences are target sequences. The “target sequence” refers to sequence being selected as the target object before the experiment. When the nucleic acid complex is chromatin, the nucleic acid segments can be located on the same chromosome or different chromosomes.

The term “interactions between nucleic acid segments” refers to the direct contact or binding of a nucleic acid segment with another nucleic acid segment by folding into a higher-order structure such as a loop; or a nucleic acid segment binds to a specific intermediary molecule (such as a protein), and the intermediary molecule also directly contacts or binds to another one or more nucleic acid segments; or a nucleic acid segment binds to a first intermediary molecule (such as a protein), and the intermediary molecule directly contacts or binds to a second intermediary molecule (such as a protein) to which one or more nucleic acid segments are bound, thereby achieving nucleic acid interactions between segments.

The term “in the nucleic acid segment” means that the recognition site of a restriction enzyme is located between the two ends of the nucleic acid segment (including the endpoints).

The term “near the nucleic acid segment” means that the recognition site of a restriction enzyme is located within a certain distance outside the two ends of the nucleic acid segment, the specific range may be 1-500 bp, 50-450 bp, 100-400 bp, 150-350 bp or 200-300 bp, preferably 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp or 350 bp.

The term “higher-order structure of genetic material” refers to the complicated three-dimensional configuration formed by helix, sheet and winding, such as chromatin or chromosome, through the interaction of DNA or RNA with proteins such as histone.

The term “genetic regulatory sequence” refers to regulatory sequences related to the structure and expression of genetic material, which may include promoters, enhancers, silencers, and other sequences capable of interacting with binding proteins having regulatory functions.

The term “other nucleic acid segments” refers to nucleic acid segments that differ from regulatory sequences and may interact with genetic regulatory sequences.

The term “sample” may be any physical subject containing DNA, and the DNA is or capable of being cross-linked. The sample may be or may be derived from biological materials.

The sample may be or may be derived from one or more cells, one or more nuclei, one or more tissues. The subject may be or may be derived from any subject that contains nucleic acids, such as chromatin. The sample may be or may be derived from one or more isolated cells or one or more isolated tissues, or one or more isolated nuclei.

The sample may be or may be derived from living cells and/or dead cells and/or nuclear lysates and/or isolated chromatin.

The sample may be or may be derived from cells of a diseased and/or non-diseased subject.

The sample may be or may be derived from a subject suspected of having a disease.

The sample may be or may be derived from a subject who is tested for the possibility of disease in the future.

The sample may be or may be derived from surviving or non-surviving patient material.

The term “cross-linking” refers to the process of fixing nucleic acids or nucleic acids with other molecules, such as proteins, using a cross-linking agent. Two or more nucleic acid segments may be cross-linked by a cross-linking agent, or the cross-linking agent may be used to cross-link the nucleic acid segments with proteins. In the present invention, cross-linking agents different from formaldehyde can be used, including those that directly crosslink nucleic acid sequences. Examples of cross-linking agents include, but are not limited to, UV light, mitomycin C, nitrogen mustard, melphalan, 1,3-butadiene diepoxide, cisdiamine dichloroplatinum (II), and cyclophosphamide.

The term “in situ cross-linking” belongs to a form of cross-linking, which means that after cross-linking, the nucleic acid itself and/or other molecules bound to it, such as proteins, retain position information as before cross-linking, or interact and relative location information.

The term “CTCF” is CCCTC binding factor, which is a transcription factor encoded by the CTCF gene. CTCF protein plays an important role in the imprinting control region (ICR) and differentially-methylated region-1 (DMR1) and MAR3 binding to inhibit the insulin-like growth factor 2 (Igf2) gene. The binding of CTCF with the target sequence can block the interaction between the enhancer and the promoter, thereby limiting the activity of the enhancer. In addition to blocking the enhancer, CTCF can also act as a chromatin barrier to prevent heterochromatin, and the human genome has nearly 15,000 CTCF sites. In addition, CTCF has multiple functions in gene regulation, and CTCF binding sites can also be used as nucleosome positioning sites.

The term “bridge linker” refers to the adaptor sequence connecting the ends of different fragments after digestion.

The term “one-step ligation” means that the ends of different nucleic acid fragments are directly connected without a linker. Therefore, free nucleic acid sequences in the reaction environment may also be linked randomly.

The term “two-step ligation” refers to connecting the ends of different nucleic acid sequences that are close in space after digestion by an adaptor (the “bridge linker” of the present invention), reducing the random collision of nucleic acid sequences in the reaction environment and reducing the free the connection of the interference sequence and the target sequence, thereby increasing the specificity.

The term “restriction enzyme” is also referred to as “restriction endonuclease” in the present invention. Restriction enzyme cuts sugar-phosphate backbone of DNA. In most cases, a given restriction enzyme recognizes and cleaves double-stranded DNA that contains several special bases.

The term “recognition site” refers to a nucleoside segment recognized by a restriction enzyme on its substrate. The sequence and length of the recognition site vary with different restriction enzymes. The length of the recognition site sequence determines to a certain extent the cleavage frequency of the enzyme in the DNA and the distance between the cleavage sites. The cleavage site may be located inside the recognition site, or several nucleotides outside the recognition site, depending on the type of enzyme. For example, in the present invention, the recognition site of HaeIII is GGCC, and its cleavage site is located inside the recognition site; and the recognition site of Mnl1 is CCTC, and its cleavage site is outside the recognition site.

“BL-Hi-C” is Bridge-Linker-Hi-C, and the name is used in the Examples section to refer to the method of the present invention, but it is not limited to the specific steps listed in the examples. It can be broadly defined as the methods of all aspects of the invention.

The term “Paired-End Tags (PETs)” refers to specific nucleic acid sequence fragments obtained after sequencing. In the present invention, the sequences of the ligation products of two or more nucleic acid segments can be determined through sequencing, that is, through PETs.

### EXAMPLES

### Example 1 Standard BL-Hi-C Method (HaeIII Enzyme and Two-Step Ligation)

### 1. Crosslinking

Mammalian K562 cells (5×10⁴ to 5×10⁵) were cultured in RPMI 1640 medium supplemented with 10% fetal bovine serum, at 37° C. and 5% CO₂. After counting the cells by an automatic counter, cells were centrifuged at 300×g for 5 minutes. The cell pellet was washed once with 1× PBS. The cells are then resuspended in fresh medium or PBS at a density not exceeding 1.5×10⁶/ml. 37% formaldehyde solution was added to the medium or PBS to a final concentration of 1% v/v, and the mixture was shaken at room temperature for 10 minutes. 2.5M glycine solution was quickly added to the mixture to a final concentration of 0.2M, and the mixture was shaken at room temperature for 10 minutes followed by ice bath for 5 minutes to terminate the cross-linking reaction. The cells were then centrifuged at 300×g for 5 minutes and washed twice with 1× PBS to separate the cross-linked cells. The isolated cells obtained can be stored at −80° C. for up to 1 year.

### 2. Cell Lysis

BL-Hi-C lysis buffer I (50 mM HEPES-KOH pH 7.5, 150 mM NaCl, 1 mM EDTA, 1% Triton X-100, 0.1% sodium deoxycholate and 0.1% SDS) containing protease inhibitor (Complete Protease Inhibitor Cocktail Tablets, Roche Applied Science, Mannheim, Germany) was added to the cells for lysis, treated at 4° C. for 15 minutes, and then centrifuged at 800×g for 5 minutes. The above steps were repeated once. The nuclei were then further treated with BL-Hi-C lysis buffer II (50 mM HEPES-KOH pH 7.5, 150 mM NaCl, 1 mM EDTA, 1% Triton X-100, 0.1% sodium deoxycholate and 1% SDS) containing protease inhibitor, at 4° C. for 15 minutes, followed by centrifugation at 3,000×g for 10 minutes. Finally, the nuclei were washed once with BL-Hi-C lysis buffer I containing protease inhibitors and frozen at −80° C.

### 3. Digestion, Ligation and DNA Purification

At 62° C., the nuclei were resuspended in 50 μl of 0.5% SDS solution for 10 minutes, 145 μl of double-distilled water was added, and 10% Triton-X100 was added to a final concentration of 1% v/v, and treatment was performed at 37° C. for 15 minutes. 25 μl 10× NEBuffer 2 and 100 U HaeIII restriction enzyme were added (New England Biolabs, Ipswich, Mass., USA, R0108L), shaken (Thermomixer comfort, eppendorf 900 rpm), 37° C. overnight (at least 2 hours). After digestion, 2.5 μl of 10 mM dATP solution and 2.5 μl of Klenow fragment (3′ to 5′exonuclease) (New England BioLabs, M0212L) were added, and incubated at 37° C. for 40 min for adding A at the end of DNA. Then, ligation buffer (750 μl ddH₂O, 120 μl 10× T4 DNA ligase buffer [New England BioLabs, B0202S], 100 μl 10% Triton X-100, 12 μl 100× BSA [New England BioLabs, B9001S], 5 μl T4 DNA ligase [New England BioLabs, M0202L] and 4 μl 200 ng/μl bridge linker) were added and shaken at 16° C. for 4 hours for two-step ligation. The obtained ligation product was centrifuged at 3500×g for 5 minutes at 4° C. The nuclei were resuspended in exonuclease mixed buffer (309 μl ddH₂O, 35 μl Lambda exonuclease buffer [New England BioLabs, B0262L], 3 μl Lambda exonuclease [New England BioLabs, B0262L], 3 μl exonuclease I [New England BioLabs, B0293L]), and was shaken at 37° C. for 1 hour to remove free bridge linkers. To reverse cross-linking, 45 μl of 10% SDS and 55 μl of 20 mg/ml proteinase K (Invitrogen, 25530-015) were added, and the reaction system was incubated at 55° C. for at least 2 hours, usually overnight. Then, 65 μl of 5M NaCl (Ambion, AM9759) was added, and the reaction system was incubated at 68° C. for 2 hours. Finally, DNA was extracted using standard phenol:chloroform (pH=7.9) and ethanol precipitation, and the DNA was resuspended in 130 μl of elution buffer (Qiagen Inc., 1014612). The obtained DNA can be stored at −20° C. for up to one year.

The double-strand bridge linker is formed by annealing the following two single-strand DNAs:

forward:
(SEQ ID NO: 1)
5P-CGCGATATC/iBIOdT/TATCTGACT (iBIOdT refers to a
biotin-labeled deoxyribonucleotide T),
and
reverse:
(SEQ ID NO: 2)
5P-GTCAGATAAGATATCGCGT.

The two single-strand nucleic acids were synthesized by company, and Biotin modification was introduced during the synthesis.

### 4. Sonication and Enrichment

The DNA was broken up to an average length of 400 bp with a Covaris S220 ultrasonic machine, and was added to 2× B&W buffer (10 mM Tris-HCl, pH=7.5, 1 mM EDTA, 2 M NaCl). 40 μl M280 streptavidin magnetic beads (Life Technologies, 11205D) were added to DNA and shaken at room temperature, and adsorbed for 15 minutes. The magnetic beads were washed 5 times with 2×SSC/0.5% SDS solution and then washed twice with 1× B&W buffer.

### 5. Library Construction

M280 magnetic beads carrying DNA were resuspended with end-repaired buffer (75 μl ddH₂O, 10 μl 10× T4 DNA ligase buffer, 5 μl 10 mM dNTP, 5 μl PNK (New England BioLabs, M0201L), 4 μl T4 DNA polymerase I (New England BioLabs, M0203L), 1 μl Klenow large fragment (New England BioLabs, M0210)), shaken at 37° C. for 30 minutes. The magnetic beads were washed twice with 600 μl 1× TWB (5 mM Tris-HCl pH=7.5, 0.5 mM EDTA, 1 mM NaCl, 0.05% Tween-20) at 55° C., 2 minutes for each time. Subsequently, the magnetic beads were resuspended with A adding buffer (80 μl ddH₂O, 10 μl 10× NEBuffer 2, 5 μl 10 mM dATP, 5 μl Klenow exo⁻ (New England BioLabs, M0212)), and shaken at 37° C. for 30 min. The magnetic beads were washed twice with 600 μl 1× TWB at 55° C., 2 minutes for each time. The beads were washed with 50 μl 1× Quick Ligase Buffer (New England BioLabs, B2200S). The beads were then resuspended in Quick Ligation Buffer (6.6 μl ddH₂O, 10 μl 2× Quick Ligase Buffer, 2 μl Quick Ligase, 0.4 μl 20 μM adapter), and incubated at room temperature for 15 min. The beads were washed twice with 600 μl 1× TWB at 55° C., 2 minutes for each wach, and then washed once with 100 μl elution buffer (Qiagen Inc., Valencia, Calif., USA, 1014612). The DNA-bound magnetic beads were resuspended in 60 μl of elution buffer and divided into two, 30 μl each. One was used for subsequent PCR, and the other was stored at −20° C. as a backup.

The double-strand adaptor is formed by annealing the following two single strands:

forward:
(SEQ ID NO: 3)
5P-GATCGGAAGAGCACACGTCTGAACTCCAGTCAC;
and
reverse:
(SEQ ID NO: 4)
TACACTCTTTCCCTACACGACGCTCTTCCGATCT.

### 6. PCR Amplification and Sequencing

DNA bound to the magnetic beads was directly amplified using PCR library primers suitable for Illumina sequencers, 9-12 cycles. Then, according to standard methods, AMPure XP beads (Beckman Coulter, A63881) were used to purify DNA to select fragments of 300-600 bp. Finally, the DNA was dissolved in 20 μl ddH₂O instead of Elution Buffer. Regarding the size selection of DNA, 0.6×volume of AMPure XP beads were added and separated by magnetic force, and the supernatant was collected. Then, 0.15×volume of AMPure XP beads were added, and the beads were collected after magnetic separation. The beads were washed twice with freshly prepared 70% ethanol and eluted with 50 μl of elution buffer (Qiagen Inc., 1014612). By using Qubit, Agilent 2100, and performing qPCR quality control, the BL-Hi-C library was sequenced using Hiseq 2500 (Illumina) (125 bp end pairing module) or Hiseq X Ten (Illumina) (150 bp end pairing module). The library PCR primers suitable for Illumina sequencer are as follows:

common primer:
(SEQ ID NO: 5)
AATGATACGGCGACCACCGAGATCTACACTCTTTCCCTACACGAC,
and
index primer:
(SEQ ID NO: 6)
CAAGCAGAAGACGGCATACGAGATCGTGATGTGACTGGAGTTCAGACGT
GT.

### 7. Data Analysis

Data was processed using ChIA-PET2 software including the removal of bridge linkers, the alignment of sequencing reads to the genome, the generation of paired-end tags (PETs) and the removal of PCR duplications.

The parameters of the two-step ligation are as follows: -m 1 -k 2 -e 1 -A ACGCGATATCTTATC -B AGTCAGATAAGATAT; and the parameters for one-step ligation are as follows: -m 2 -k 2 -e 1 -A AGCTGAGGGATCCCT -B AGCTGAGGGATCCCT.

The obtained PETs can be used for downstream interaction matrix construction, hot map analysis, protein binding peak and read cluster analysis.

The following steps 8-10 are optional according to different experimental needs.

### 8. BL-Hi-C Enrichment Analysis

The PETs obtained by BL-Hi-C and the PETs obtained by in situ Hi-C in public databases are converted into bed format files for enrichment analysis, or rmdup.bedpe.tag output files that can be directly processed by ChIA-PET2 software. Use bedtools software to find the PETs that overlap with the public database chromatin immunoprecipitation (ChIP-seq) peaks by the command “bedtools intersect -u”. For BL-Hi-C and in situ Hi-C (Rao et al.), the ChIP-seq data in public database from CTCF and RNAPII on K562 cell line is used; for HiCHiP method, the data in public database from GM12878 cell line is used; for in situ Hi-C (Nagano et al.), data from the H1hesc cell line is used. The same strategy is also applicable to the analysis of ChromHMM annotation. ENCODE processes the “bam” files for the input, and the overlapping from the CTCF and RNAPII ChIP-seq data is used to show the enrichment pattern. Then, the bedtools command “bedtools coverage -sorted” is applied to calculate the depth for each group of CTCF or RNAPII peaks. In addition, the homer software command “annotatePeaks.pl” is used to calculate the enrichment of genomic features for each group.

### 9. BL-Hi-C Loop Analysis

The common loops are identified using the bedtools software command “bedtools pairtopair -type both”. In addition, the others are grouped into specific loops. For CTCF motif orientation analysis, the contacts with a single CTCF motif obtained from the ENCODE motif repository are used to calculate the proportions of convergent, divergent, or identical orientation. For the heatmap analysis, the contact matrixes of BL-Hi-C and in situ Hi-C are normalized by sequencing depth and then converted into differential heatmaps. For visual 4C analysis, the interactions are extracted from the original PET file. Then, MICC software is applied to generate PET clusters and calculate the depth and interaction counts for the clusters, which are further visualized by the WashU Epigenome Browser.

### 10. Models Analysis

The BL-Hi-C data Are processed directly with ChIA-PET2 to obtain the PETs and peaks using the following command: -m 1 -t 4 -k 2 -e 1 -1 15 -S 500 -A ACGCGATATCTTATC -B AGTCAGATAAGATAT -M “--nomodel -q 0.05-B --SPMR --call-summits” for the two-step ligation data and -m 2 -t 4 -k 2 -e 1 -1 15 -S 500 -A AGCTGAGGGATCCCTCAGCT -B AGCTGAGGGATCCCTCAGCT -M “--nomodel -q 0.05 -B --SPMR --call-summits” for the one-step ligation data. Then, the depth per 1 M sequencing reads for each peak is calculated and converted the bed file into a bedgraph file with the command “bedGraphToBigWig”. “ComputeMatrix” software is then used to calculate the distance distribution for the enzyme comparison. Here the samples cut by HaeIII are randomly sampled to a depth of 35 M PETs to make them comparable to the samples cut by MboI or HindIII.

### Example 2 BL-Hi-C Using MboI or HindIII and Two-Step Ligation Method

Cross-linking, cell lysis, DNA purification, sonication and enrichment, library construction, PCR amplification and sequencing are the same as the standard BL-Hi-C protocol in Example 1. The digestion and ligation steps are as follows. The nuclei were gently resuspended in 50 μl 0.5% SDS and incubated at 62° C. for 10 minutes. Then, the mixture was added with 145 μl ddH₂O and 10% Triton-X100 (final concentration of 1% v/v), and incubated at 37° C. for 15 minutes. 25 μl 10× NEBuffer 2 and 100 U MboI or HindIII restriction enzyme (New England BioLabs, R0147L or R3104L) were added, and shaken overnight at 37° C. (Thermomixer comfort, eppendorf 900 rpm), and then heated at 62° C. for 20 minutes. 36 μl ddH₂O, 1.5 μl 10 mM dNTP, 8 μl Klenow large fragment (New England BioLabs, M0210) were added to the mixture and shaken at 37° C. for 45 minutes. Then, the cell nuclei were centrifuged at 2000×g for 5 minutes, 250 μl ddH₂O, 25 μl NEBuffer 2, 2.5 μl 10 mM dATP solution (New England BioLabs, M0212L) and 2.5 μl Klenow fragment (3′ to 5′exo−) (New England BioLabs, M0212L) were added and shaken at 37° C. for 40 minutes in order to add A tail. The subsequent steps are the same as the standard BL-Hi-C protocol in Example 1.

### Example 3 BL-Hi-C Using HindIII and One-Step Ligation Method

Cross-linking, cell lysis, DNA purification, sonication and enrichment, library construction, PCR amplification and sequencing are the same as the standard BL-Hi-C protocol in Example 1. For the ligation, ligation buffer (735 μl ddH₂O, 120 μl 10× T4 DNA ligase buffer [New England BioLabs, B0202S], 100 μl 10% Triton X-100, 12 μl 100× BSA [New England BioLabs, B9001S], 5 μl T4 DNA ligase [New England BioLabs, M0202L] and 20 μl of 90 ng/μl half bridge linker were added and shaken at 16° C. for 4 hours for one-step ligation. The obtained ligation product was centrifuged at 4° C. 3500×g for 5 minutes. Subsequently, the nuclei were added with 170 μl ddH₂O, 20 μl 10× T4 DNA ligase buffer, 10 μl T4 PNK (New England BioLabs, M0201L), and shaken at 37° C. for 1 hour. The obtained product was centrifuged at 3500×g at 4° C. for 5 minutes, and then added with the ligation buffer (755 μl ddH₂O, 120 μl 10× T4 DNA ligase buffer, 100 μl 10% Triton X-100, 12 μl 100× BSA, 5 μl T4 DNA Ligase) for resuspending, and shaken at 16° C. for 4 hours for one-step ligation. The ligated product was centrifuged at 3500×g for 5 minutes at 4° C., and then the nuclei were suspended in the same exonuclease mixing buffer as the standard BL-Hi-C protocol. The double-strand half bridge linker is formed by annealing two single strands (forward: 5P-GCTGAGGGA/iBiodT/C; reverse: CCTCAGCT).

### Example 4 Comparison of In Situ Hi-C and HiChI

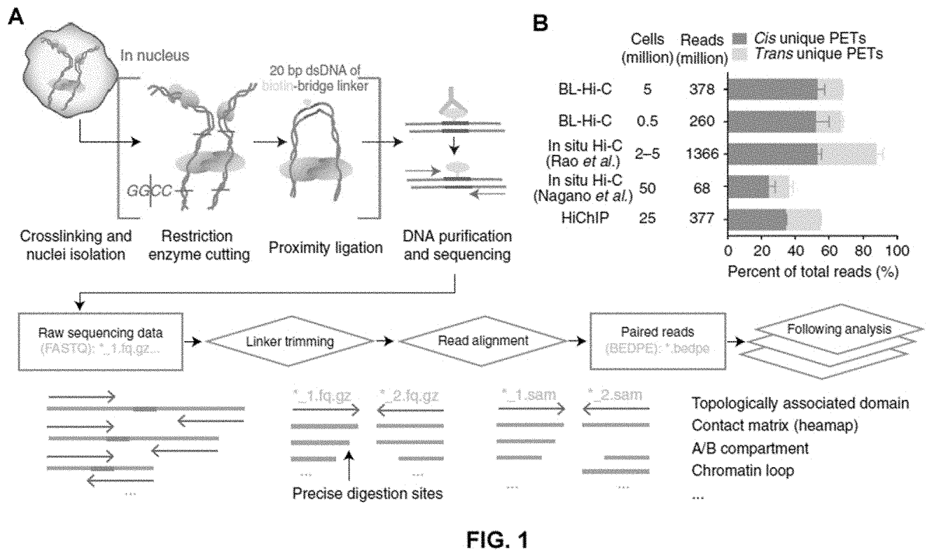

Compare the method of Example 1 (see FIG. 1-A for the overall process) with the published in situ Hi-C and HiChIP methods. The results show that more than 60% of the total sequenced reads were joined into unique PETs for BL-Hi-C, which reflected greater efficiency than that of the in situ Hi-C22 and HiChIP13 methods (FIG. 1-B). The ratio of cis- and trans-unique PETs, which is generally considered to relate to the signal-to-noise ratio, was 5.83±0.29 for BL-Hi-C, 2.10±0.98 for in situ Hi-C21, and 3.85±0.18 for HiChIP13. BL-Hi-C of Example 1 presents higher efficiency for unique PET formation and higher confidence in cis-unique PET detection.

### Example 5 Enrichment of Sequences for DNA Binding Proteins

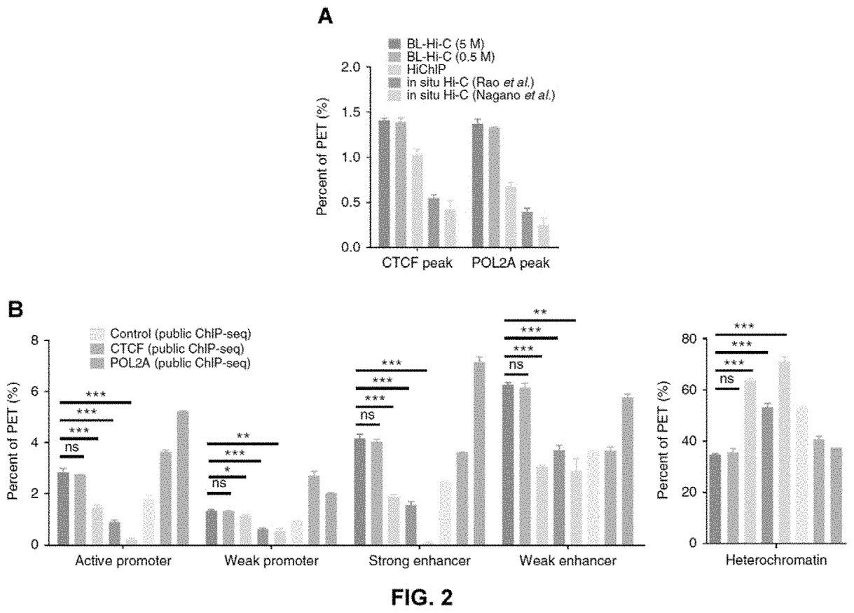

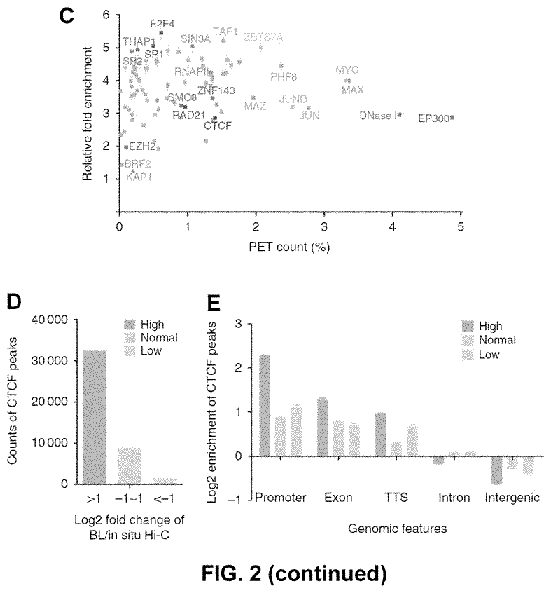

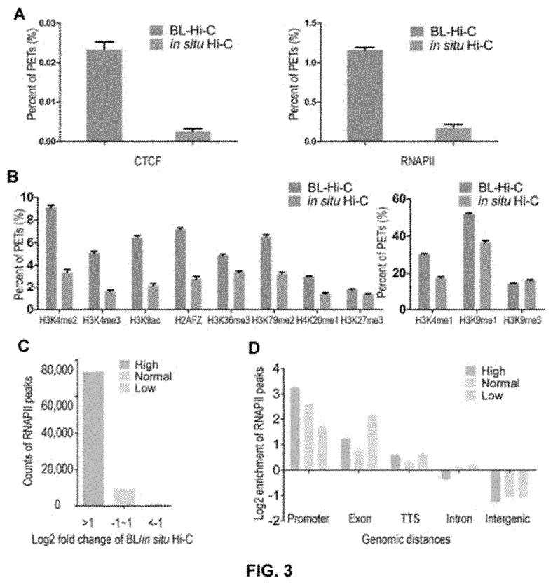

CCCTC-binding factor (CTCF) and RNA polymerase II (RNAPII) play important roles in regulating the genome architecture and enhancer-promoter interactions. CTCF and RNAPII ChIP-seq peaks in chromatin interaction anchor regions are examined. It is found that there are about 1.3 to 3.3-fold CTCF enrichment and about 2.0 to 5.4-fold RNAPII enrichment for BL-Hi-C PETs compared to in situ Hi-C and HiChIP (FIG. 2-A and FIG. 3-A).

Furthermore, BL-Hi-C PETs are mapped to chromatin regions annotated by ChromHMM with public hi stone ChIP-seq data sets. Compared with in situ Hi-C, there are more than 3-fold the number of BL-Hi-C PETs detected at active promoters and strong enhancers, while <50% of the number of interactions are detected at heterochromatin regions (FIG. 2-B and FIG. 3-B). Notably, the BL-Hi-C enrichment pattern is comparable to that of ChIP-seq captured by CTCF or RNAPII, strongly indicating that BL-Hi-C dramatically enriches PETs at CTCF or RNAPII-binding regions.

Moreover, BL-Hi-C PETs have about 1 to 5-fold enrichment at TF-binding sites annotated by the ChIP-seq peaks of 83 TFs in the K562 cell line, suggesting a global enrichment of BL-Hi-C (FIG. 2-C). Furthermore, to investigate the specificity of BL-Hi-C enrichment, CTCF or RNAPII ChIP-seq peaks are classified into groups according to the depth accumulated with the normalized PETs of the BL-Hi-C or the in situ Hi-C method. For BL-Hi-C, high, normal, and low corresponded to log2-fold changes of depth >1, between 1 and −1, and >−1, respectively (FIG. 2-D and FIG. 3-C).

The distributions of these grouped peaks of CTCF and RNAPII are examined with respect to genomic features 25. It is found that the peaks of BL-Hi-C are significantly enriched at promoters but not enriched at introns and intergenic regions (FIG. 2-E and FIG. 3-D). Taken together, BL-Hi-C is an enrichment method that is more efficient at capturing regulatory protein-binding sites than either in situ Hi-C or HiChIP, especially in the active euchromatin regions.

### Example 6 Influence of Different Restriction Enzymes (HaeIII, MboI and HindIII) on the Results

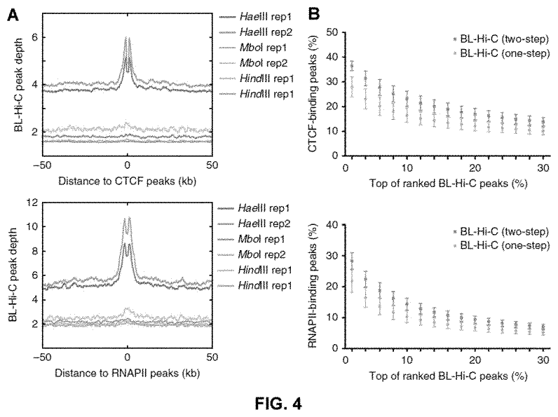

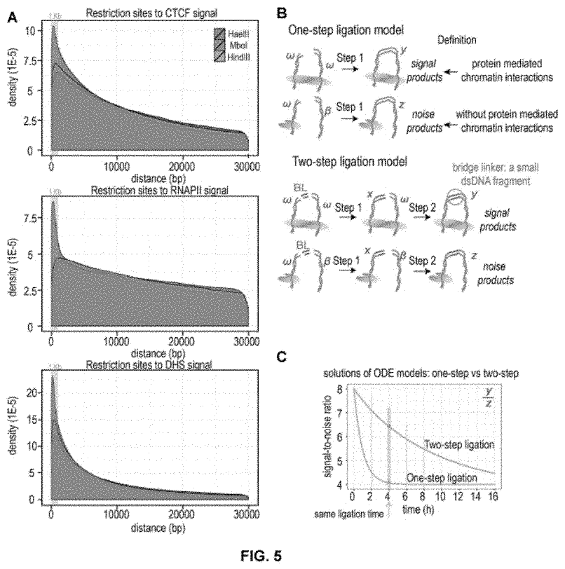

As shown in Example 2, HaeIII, MboI and HindIII were used in parallel in the two-step ligation. The sequencing data were converted into peaks and studied the distance distribution between BL-Hi-C peaks and public ChIP-seq peaks such as CTCF or RNAPII. The results strongly demonstrate that the genomic break points generated by HaeIII are enriched and within ±1 kb of the DNA-binding proteins for both CTCF and RNAPII, but the break points generated by MboI and HindIII are not enriched, indicating that enzyme digestion can significantly increase the sensitivity of protein-centric chromatin interaction detection (FIG. 4-A and FIG. 5-A).

### Example 7 Comparison of One-Step Ligation and Two-Step Ligation

In the model based on two-step ligation (FIG. 5-B), DNA fragments that are pulled closer by specific protein complexes will be more preferentially ligated with bridge linkers; compared to one-step ligation, the two-step ligation method amplifies this advantage (FIG. 5-C). Subsequently, as in Example 3, the HaeIII was used for digestion, and the sequencing data was converted into peaks to detect whether there was protein binding. Comparing the results of the one-step ligation method and the two-step ligation method, it can be found that more CTCF and RNAPII binding peaks were detected by the two-step ligation, indicating that the two-step ligation mediated by the bridge linker reduces the random connection of DNA and increases the detection specificity of protein-mediated chromatin interaction (FIG. 4-B).

### Example 8 Compared with In Situ HiC, BL-Hi-C Can Detect More Chromatin Loops

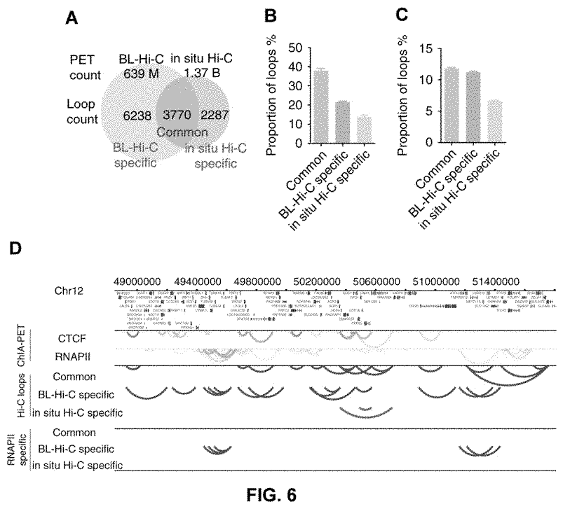

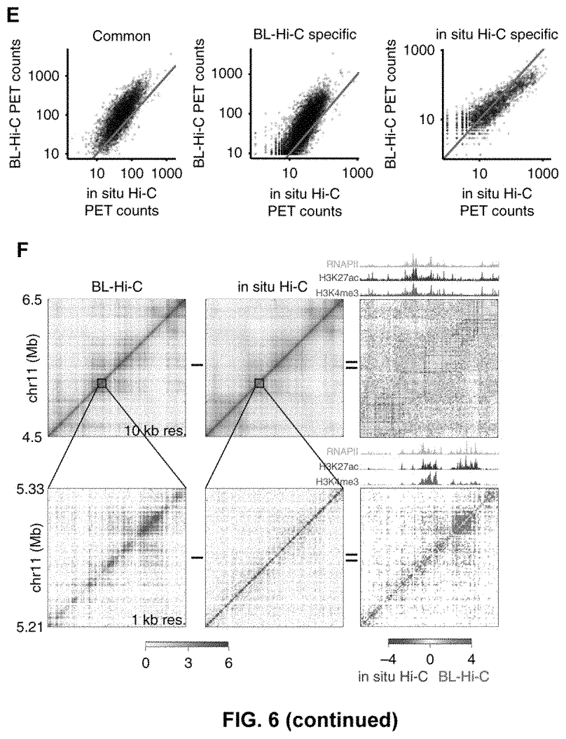

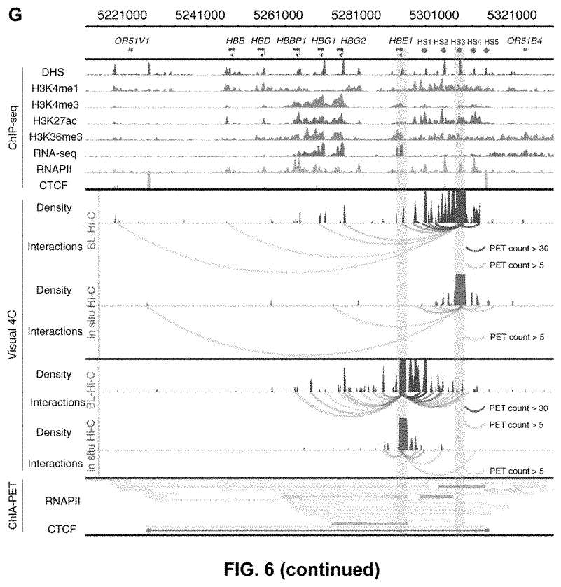

10,014 loops from 639M reads were identified by BL-Hi-C, which is much more efficient than in situ Hi-C, which identified 6,057 loops from 1.37 B reads. Further, the loops were grouped into common loops detected by both methods and specific loops detected only by BL-Hi-C or only by in situ Hi-C (FIG. 6-A). The results show that there are more CTCF and RNAPII ChIA-PET loops among the loops detected by BL-Hi-C than among those detected by in situ Hi-C (FIG. 6-B and FIG. 6-C). Meanwhile, the common loops are frequently overlapped with the CTCF ChIA-PET loops (possibly representing more invariant architectures), but the BL-Hi-C-specific loops are often overlapped with the RNAPII ChIA-PET loops, as illustrated for a typical region in FIG. 6-D.

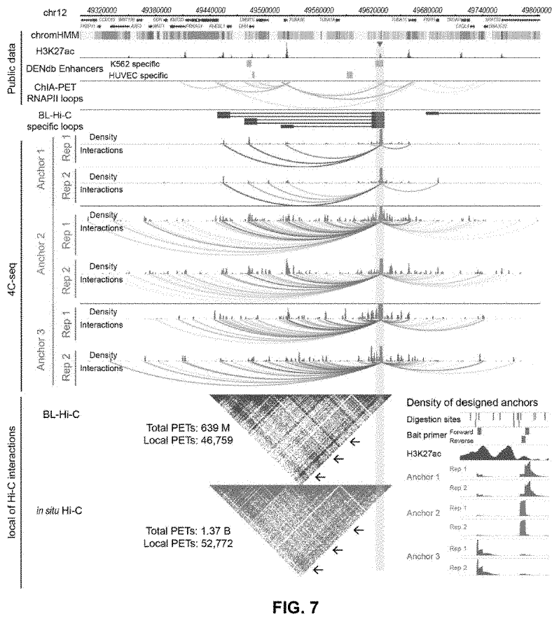

To verify the chromatin loops identified specifically by the BL-Hi-C method, 4C-seq was performed on the illustrated region (FIG. 7). The results showed that the BL-Hi-C loop anchors are consistent with the 4C-seq anchors, the H3K27ac signals, and the cell-specific enhancers collected by DENdb26. In addition, the 4C-seq-validated chromatin interaction regions showed higher signal-to-background ratios for BL-Hi-C than for in situ Hi-C. At the whole-genome level, the results are consistent with those in the local region, in that BL-Hi-C produced more contact counts in the commonly detected loop regions than did in situ Hi-C (FIG. 6-E). These results revealed that BL-Hi-C is more sensitive for the detection of structural and regulatory loops.

The beta-globin region in chromosome 11 was chosen for analysis, and the contact maps were shown at 10-and 1-kb resolution (FIG. 6-F). It was found that the BL-Hi-C signals are highly correlated with active histone modifications, such as H3K27ac and H3K4me3. Upon close inspection of the beta-globin region (FIG. 6-G), it was found that HS3 was most active in 5LCR regions, and is connected more closely with the active HBE1 and HBG promoters than with the repressed HBB and HBD genes, which is consistent with the previous RNAPII ChIA-PET loops studies. Importantly, with only half of the sequencing depth, BL-Hi-C method detected 3.1-fold more functional chromatin interactions on average than did in situ Hi-C.

### Example 9 More Endonuclease Selection and Analysis

The information storage unit of human genome information is a linear combination of four bases, AGCT. Theoretically, there are 256 combinations of recognition sites with consecutive four-base sequences, and 4096 combinations for recognition sites with consecutive six-base sequences. Therefore, if the bases of the genome are ideally evenly distributed, a specific continuous four-base sequence recognition site can appear every 256 bp, and a specific continuous six-base sequence recognition site can appear on an average of 4096 bp. Therefore, an enzyme that recognizes four bases has a higher digestion resolution than an enzyme that recognizes six bases.

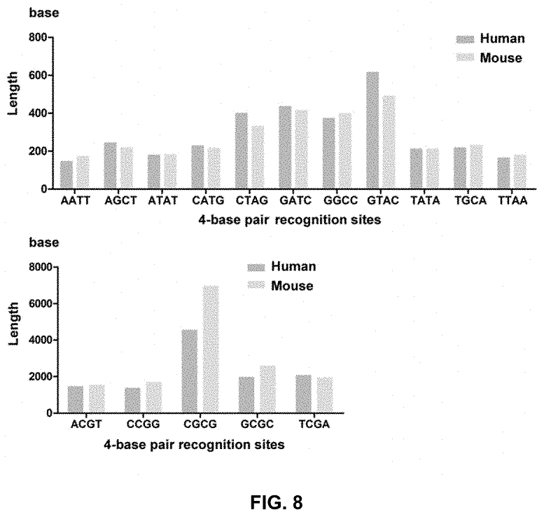

In order to more accurately study the actual distribution of different four-base restriction endonuclease sites, the human genome and mouse genome were selected for analysis. The human genome uses the hg19 version. The total length of 22 autochromosomes plus X and Y chromosomes is 3,095,677,412 bp; the mouse genome uses the mm 9 version. The total length of 19 euchromatins plus X and Y chromosomes is 2,654,895,218 bp. The type II restriction endonuclease recognition sites were used as the analysis object, covering 16 four-base recognition sites (FIG. 8). It was found that the distribution of four-base recognition sites in the genome was very different. The average length of the seven four-base recognition sites of AATT, AGCT, ATAT, CATG, TATA, TGCA and TTAA in the genome is less than the theoretical value 256 bp; and the average length of ACGT, CCGG, CGCG, GCGC and TCGA four-base recognition sites in the genome is more than four times the theoretical value of 256 bp. This reflects the impact of the actual heterogeneity of the genome on the digestion result.

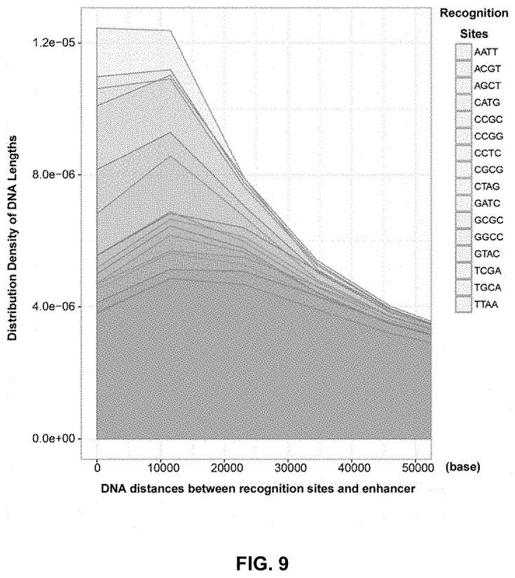

Next, the distribution of four-base recognition sites on promoters and enhancer elements was studied. It was found that the distribution of CTAG, GTAC, GGCC, CGCG, CCTC and CCGG, five endonuclease recognition sites, is significantly close to the distribution of promoters and enhancers on the genome (FIG. 9).

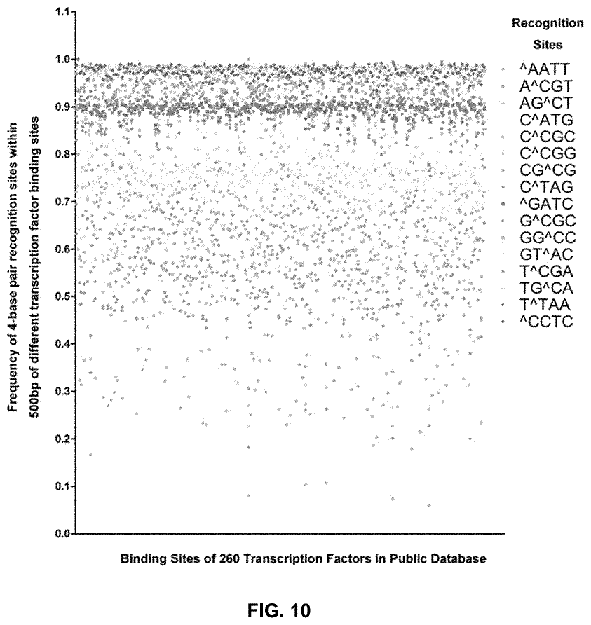

Subsequently, the distribution of four-base endonuclease recognition sites within five hundred bases of different transcription factor binding sites in the K562 cell line was studied. The results show that the frequency of the same restriction endonuclease recognition site near different transcription factor binding sites is relatively stable, and there is a big difference only in a few transcription factor binding sites. Among them, the four restriction endonuclease recognition sites of CCTC, TGCA, GGCC, and AGCT appear frequently within the five hundred bases of transcription factor binding sites, with an average frequency of over 95%; CATG, AATT, CTAG and GATC within five hundred bases of the transcription factor binding site, with a frequency of over 90%; while the frequency of CGCG, TCGA, GCGC and CCGC within 500 bases of transcription factor binding sites is low, not more than 70% (FIG. 10).

What is claimed is:

1. A method of analyzing interactions between two or more nucleic acid segments in a nucleic acid complex, comprising
1) providing a sample comprising the nucleic acid complex;
2) exposing the nucleic acid complex obtained in step 1) to a restriction enzyme of which the recognition site is located in or near at least one of the nucleic acid segments, and performing digestion;
3) subjecting the resultant of the digestion from step 2) to ligation; and
4) identifying the sequences of the two or more nucleic acid segments which are ligated in step 3).

2. The method according to claim 1, wherein the sample in step (1) is a sample after cross-linking treatment.

3. The method according to claim 2, wherein the cross-linking treatment is performed by using cross-linking agent, specifically, the cross-linking agent is selected from the group consisting of glutaraldehyde, formaldehyde, epichlorohydrin and toluene diisocyanate, preferably formaldehyde; optionally, the cross-linking is in situ cross-linking.

4. The method according to claim 1, wherein the two or more nucleic acid segments are genetic regulatory sequences, preferably, the genetic regulatory sequences are promoter, silencer and enhancer; wherein the two or more nucleic aide segments are bound to one or more binding proteins, which are preferably selected from transcription factor, enhancer binding protein, RNA polymerase and/or CTCF.

5. The method according to claim 1, wherein the restriction enzyme is a restriction enzyme with a recognition site of four-base sequence, preferably a restriction enzyme with a recognition site of GGCC and/or CCTC, and more preferably HaeIII or MnlI.

6. The method according to claim 1, wherein the ligation in step 3) is performed by using bridge linker to link the nucleic aide segments after digestion, specifically,
the bridge linker is an adaptor sequence capable of linking the terminals of different nucleic aide segments;
the bridge linker is a double-stranded nucleic acid;
the length of the bridge linker is 10-60 bp, 15-55 bp, 20-50 bp, 25-45 bp or 30-40 bp, such as 15 bp, 16 bp, 17 bp, 18 bp, 19 bp, 20 bp, 21 bp, 22 bp, 23 bp, 24 bp, 25bp, 26 bp, 27 bp, 28 bp, 29 bp, 30 bp, 31 bp, 32 bp, 33 bp, 34 bp or 35 bp, preferably 20 bp; and
the bridge linker may be labeled with one or more markers, preferably, the marker is isotopes, biotin, digoxin (DIG), fluorescein (such as FITC and rhodamine) and/or a probe, more preferably biotin,
preferably, the marker is labeled at the 5′ terminal, 3′ terminal or middle region of the bridge linker, specifically, the marker may be labeled in any one strand or both strands of the double-stranded nucleic acid.

7. The method according to claim 1, wherein the identification of ligated sequences in step 4) is performed by sequencing, preferably, the sequencing is Sanger sequencing, second generation sequencing, single molecule sequencing and single cell sequencing, more preferably second generation sequencing; and
optionally, upon the identification of ligated sequences in step 4), the method further comprises steps of de-crosslinking, nucleic acid purification, fragmentation (e.g. by sonication), enrichment, library construction and/or PCR amplification.

8. A method of identifying nucleic aide sequence interacting with one or more genetic regulatory sequences of interest, comprising the steps of the method according to claim 1.

9. A kit for the method according to claim 1, comprising a restriction enzyme capable of recognizing GGCC and/or CCTC sites and/or bridge linkers, wherein
the restriction enzyme is capable of recognizing four bases site, preferably a restriction enzyme capable of recognizing CCTC and/or GGCC sites, more preferably HaeIII or MnlI;
the length of the bridge linker is 10-60 bp, 15-55 bp, 20-50 bp, 25-45 bp or 30-40 bp, such as 15 bp, 16 bp, 17 bp, 18 bp, 19 bp, 20 bp, 21 bp, 22 bp, 23 bp, 24 bp, 25bp, 26 bp, 27 bp, 28 bp, 29 bp, 30 bp, 31 bp, 32 bp, 33 bp, 34 bp or 35 bp, preferably 20 bp;
the bridge linker may be labeled a marker, preferably, the marker includes a biotin, fluorescein and antibody, more preferably biotin; preferably, the biotin is added during the strand synthesis of the bridge linker;
preferably, the marker is labeled at the 5′ terminal, 3′ terminal or middle region of the bridge linker; and
optionally, the kit is a kit for sequencing or library construction.
